(19) United States
(12) Patent Application Publication
(10) Pub. No.: US 2013/0158499 A1
(43) Pub. Date: Jun. 20, 2013

(54) ADJUSTABLE DIAPER AND METHODS OF USING SAME

(71) Applicant: Stacy Thompson, Albion, IL (US)

(72) Inventor: Stacy Thompson, Albion, IL (US)

(21) Appl. No.: 13/525,077

(22) Filed: Jun. 15, 2012

Related U.S. Application Data
(60) Provisional application No. 61/497,349, filed on Jun. 15, 2011.

(51) Int. Cl. A61F 13/49

(52) U.S. Cl. CPC A61F 13/49003
USPC 604/385.14

(57) ABSTRACT

In accordance with some embodiments, a diaper includes a waist portion, a crotch portion, an inner surface, and an outer surface. The waist portion has a front portion and a back portion. The waist portion is configured to be positioned about the waist of an individual. The crotch portion is positioned between the front and back portions of the waist portion. The crotch portion is configured to be positioned about the crotch of the individual. The inner surface of the diaper is provided with a plurality of fasteners so as to adjust a rise of the diaper to fit the individual.

### CROSS-REFERENCE TO RELATED APPLICATIONS

The present application claims the benefit under 35 U.S.C. §119(e) of U.S. Provisional application Ser. No. 61/497,349, filed Jun. 15, 2011, entitled “Adjustable Diaper and Methods of Using Same”, which is hereby expressly incorporated herein in its entirety.

### STATEMENT REGARDING FEDERALLY SPONSORED RESEARCH OR DEVELOPMENT

Not applicable.

### FIELD OF THE INVENTION

The present invention relates generally to diapers, and more particularly, but not by way of limitation, to an improved reusable diaper having fasteners which adjust to the size of a child.

### BACKGROUND OF THE INVENTION

Various types of diapers are currently produced and sold on the market. Typically, diapers are designed to fit children in various stages of growth from newborn to toddler. Diapers are usually characterized as cloth or disposable and are constructed from absorbent materials such as cotton, hemp, bamboo, microfiber absorbent polymers, etc. Generally, diapers are constructed to fit specific ranges of body sizes based on weight. As children grow in size and weight, they outgrow one type of diaper designed for a specific range, which necessitates a switch to the next size range of diapers. For example, diapers, typically, come in five sizes: (1) up to fourteen pounds; (2) from twelve to fourteen pounds; (3) sixteen to twenty-eight pounds; (4) twenty-two to thirty-seven pounds; and (5) over thirty pounds. Once children outgrow one size range of diapers, parents are forced to purchase the next size range of diapers, which often come in packages of lower quantity and higher price.

To this end, although diapers of the existing art are usable, further improvements are desirable to enhance the usability and reusability of a diaper which functions to be adjusted as a child grows. It is to such a diaper that the present invention is directed.

### BRIEF DESCRIPTION OF THE DRAWING(S)

FIG. 1 is a perspective view of one embodiment of a diaper constructed in accordance with the present invention shown positioned about an individual.

FIG. 2 is an inner perspective view of one embodiment of the diaper of FIG. 1, in the open position, the diaper constructed in accordance with the present invention having a soaker positioned therein.

FIG. 3 is a perspective view of an inner surface of the diaper of FIG. 1.

FIG. 4 is a perspective view of the inner surface of the diaper of FIG. 1 in an adjusted position.

FIG. 5 is a perspective view of the diaper of FIG. 1 in the closed position.

### DETAILED DESCRIPTION

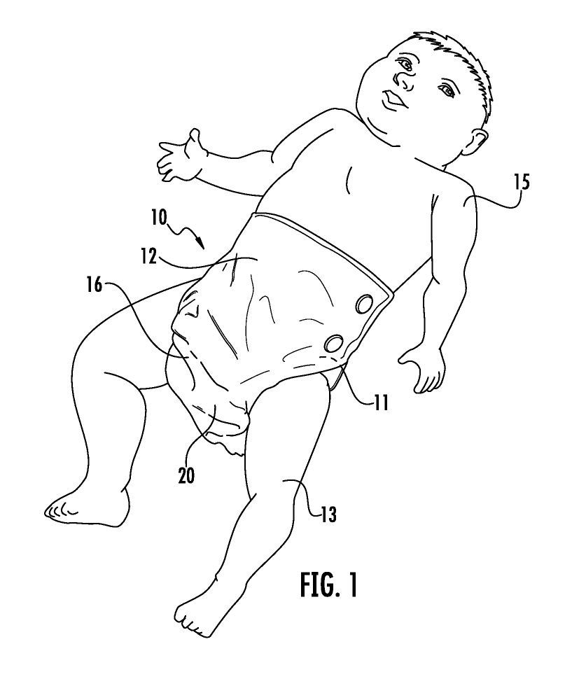

Referring now to the drawings, and more particularly to FIG. 1, shown therein is one embodiment of a diaper 10 constructed in accordance with the present invention. The diaper 10 is shown positioned about a waist 11 and legs 13 (lower torso) of an individual 15. It is contemplated that the diaper 10 is constructed from multiple reusable materials and components, however, it should be understood that the diaper 10 may be constructed from a single component. The diaper 10 is preferably made of a durable material which is strong enough to prevent damage to the diaper 10, and is absorbent and reusable. Suitable materials for construction of the diaper 10 and any components thereof include reusable absorbent materials such as cotton, hemp, bamboo, microfiber absorbent polymers, combinations thereof, etc. It should be understood that any absorbent material may be utilized with the present invention so long as the material functions in accordance with the present invention. Other materials used in the construction of the diaper may be polymeric materials, plastics, metals, such as aluminum, steel, titanium, magnesium or alloys containing these metals, woods, abrasive materials, permeable materials, impermeable materials, and/or composite materials which are capable of providing the desired strength and durability for the diaper 10. The material may be water-resistant or waterproof so that the diaper 10 may be easily cleaned and/or disinfected after use to be reused. The diaper 10 may be semi-rigid, flexible, semi-flexible, foldable, collapsible, and the like. Further, it will be appreciated that the diaper 10 can be sized from any suitable material in a manner that is positionable about, and removably attached to an individual, and/or otherwise functions as described herein. It should be understood by one of ordinary skill in the art that the diaper 10, although discussed herein as being used with a human child, may be utilized and configured in a variety of shapes and capacities to be utilized for individuals of various sizes and ages, animals, and the like.

It should be understood by one of ordinary skill in the art that the diaper 10, or portions thereof, may be any shape, such as substantially rectangular, circular, oval, square, rectangular, triangular, polygonal, quadrilateral, ellipsoidal and the like, for example. It should be understood that, in some embodiments, the size and configuration of the diaper 10, and portions thereof, may vary widely dependent upon the size of the individual for which protection is desired.

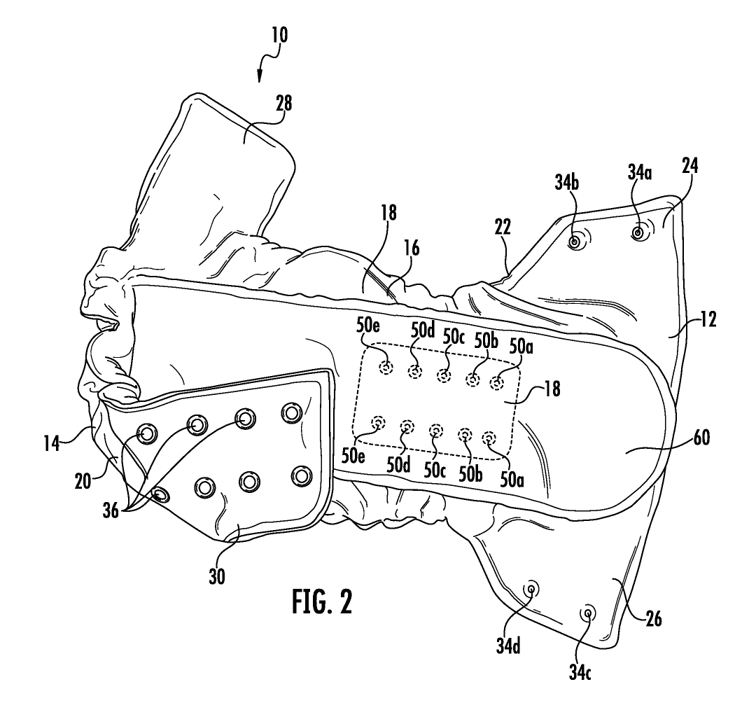

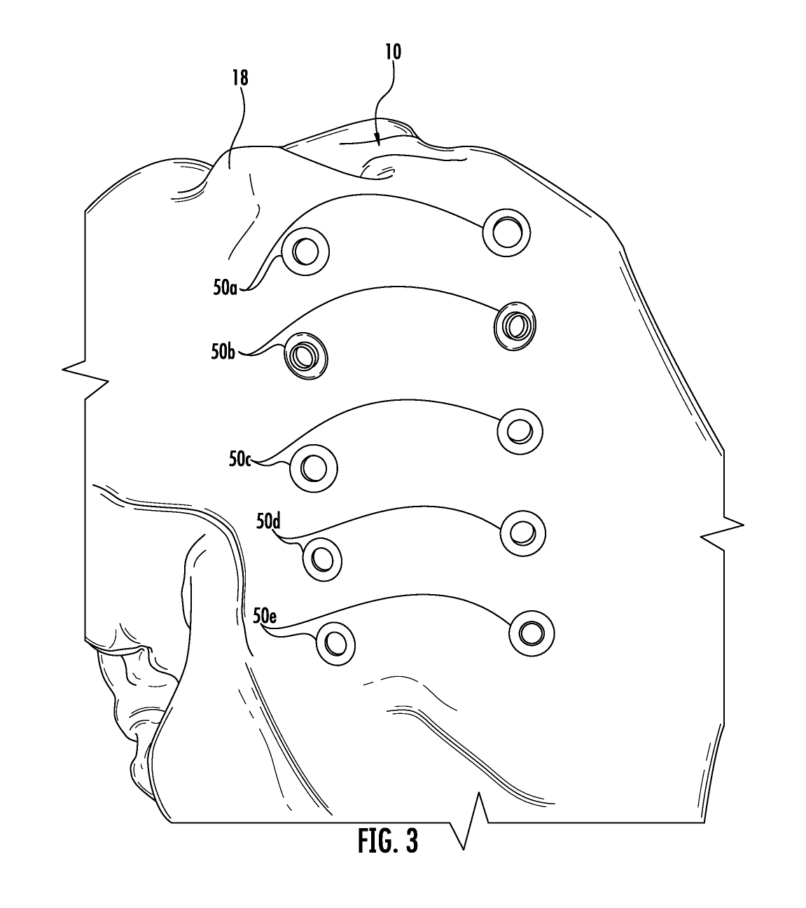

Referring to FIGS. 2-5, generally, in one embodiment, the diaper 10 includes a (first) front waist portion 12, a (second) back waist portion 14, and a crotch portion 16 positioned between the front waist portion 12 and the back waist portion 14. The crotch portion 16 is configured to be positioned about, and cover the crotch portion of the individual 15, and the front and back waist portions 12 and 14 are configured to be positioned about to cover the waist 11 of the individual 15. The diaper 10 also includes a first (inner) surface 18, a second (outer) surface 20, and an edge 22.

The front waist portion 12 includes a pair of substantially identical wings 24 and 26, generally symmetrically disposed from one another. The back waist portion 14 also includes a pair of wings 28 and 30, generally symmetrically disposed from one another. Elastic or elastic-like material may be disposed along the back waist portion 14 and the edge 22 to help ensure a snug fit of the reusable diaper 10 around the waist 11 and legs 13, respectively, of the individual 15.

A portion of the wings 24 and 26 of the front waist portion 12 may be provided with at least one fastener 34 for removably attaching the diaper 10 to the individual. A portion of the wings 28 and 30 of the back waist portion 14 may be provided with at least one fastener 36 corresponding to the fastener 34 for removably attaching the diaper 10 to the individual. The fasteners 34 and 36 may be any fastener known in the art for fastening or removably securing one object to another including, for example, standard push-button snaps, Velcro-type fasteners, straps, adhesive substances, combinations thereof, and the like. As shown in FIG. 2, in one embodiment, the wings 24 and 26 may each include a pair of fasteners 34 (labeled in FIG. 2 via the reference numerals 34a-34b and 34c-34d, respectively, for purposes of clarity). Further, the wings 28 and 30 may each include a plurality of fasteners 36 arranged and aligned in two rows. The fasteners 34a and 34b of the wing 24 correspond to attach to the fasteners 36 of the wing 28, thus attaching wings 24 and 28. Fasteners 34c and 34d of wing 26 correspond to attach to the fasteners 36 of the wing 30, thus attaching wings 26 and 30. The fasteners 36 positioned on the wings 28 and 30 may be hidden beneath the diaper 10 material on the inner surface 18 side to prevent marks on the skin of the child or individual 15. It should be understood by one of ordinary skill in the art that the fasteners 34 and 36 may be configured in any array and/or number, so long as the fasteners function in accordance with the present invention as described herein.

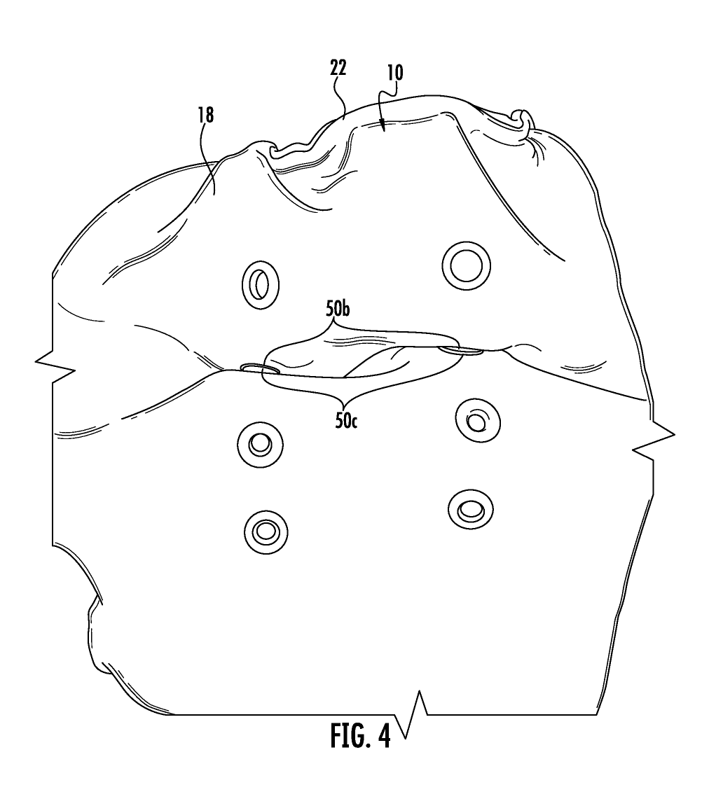

Referring now to FIGS. 2-4, a plurality of fasteners 50 (labeled in FIGS. 2-4 via the reference numerals 50a, 50b, 50c, 50d, 50e, for purposes of clarity) are provided along the inner surface 18 of the diaper 10 to allow for adjustment of the rise of the diaper 10 so that the diaper 10 may fit various-sized individuals. In one embodiment, the plurality of fasteners 50 are provided along a portion of the inner surface 18 of the crotch portion 16 of the diaper 10. However, in other embodiments, it should be understood that the plurality of fasteners 50 may be positioned along the inner surface 18 of any portion of the diaper 10. For example, such as a portion of the front and back waist portions 12 and 14, respectively, and/or, for example, any combinations thereof, so long as the plurality of fasteners 50 functions in accordance with the present invention as described herein. In one embodiment, two columns of five fasteners 50 are provided in order to adjust the rise of the diaper 10 to various-sized individuals. For example, as shown in FIG. 4, fasteners 50b may be removably attached to fasteners 50c to decrease the rise of the diaper 10 to fit a smaller individual. It should be understood that the plurality of fasteners 50 may be aligned and/or arrayed in various configurations so long as the rise of the diaper 10 may be adjusted to function in accordance with the present invention as described herein. The fasteners 50 may be color-coded to provide for identification of a range of sizes of various-sized individuals. For example, in one embodiment, fasteners 50b may be blue and fasteners 50c may be red. The fasteners 50 may be any fastener known in the art for fastening or removably securing one object to another including, for example, standard push-button snaps, Velcro-type fasteners, straps, adhesive substances, combinations thereof, and the like.

Referring to FIG. 2, in one embodiment, the diaper 10 may be provided with a soaker 60. The soaker 60 is positioned along the inner surface 18 of the diaper 10 for absorbing fluid or any other waste material from an individual. The soaker 60 may be configured, folded, and/or sized to be utilized with various sizes of the diaper 10 when the rise is adjusted as described herein. The soaker 60 may be attached to a portion of the inner surface 18 of the diaper 10 to maintain the soaker 60 in any desired position to be utilized with the diaper 10. However, it should be understood that the soaker 60 may be removably attached to any portion of the diaper 10 so long as the soaker 60 functions in accordance with the present invention as described herein. Further, it should be understood by one of ordinary skill in the art that, in other embodiments, the diaper 10 may be utilized without the soaker 60. The soaker 60 is constructed from an absorbent material, such as cotton, hemp, bamboo, microfiber absorbent polymers, combinations thereof, and the like. It should be understood by one of ordinary skill in the art that any absorbent material may be utilized with the present invention, so long as the material functions in accordance with the present invention.

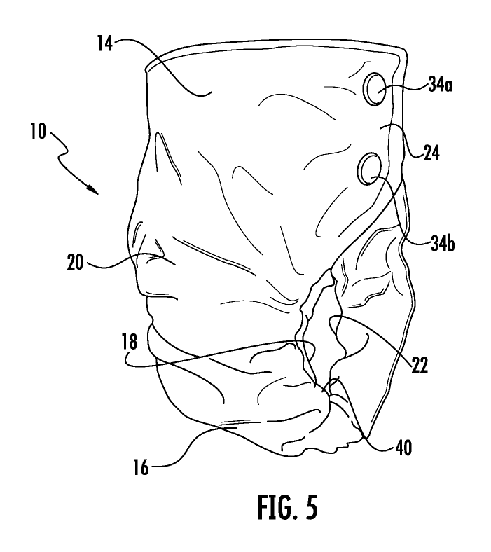

Referring to FIG. 5, in the closed position, the front waist portion 12 is removably attached to the back waist portion 14 so that the edges of the front waist portion 12 and the back waist portion 14, in cooperation with the crotch portion 16, define leg openings 40 and 42 for the legs of an individual. The leg openings 40 and 42 may include an elastic material disposed about the periphery of the leg openings 40 and 42 to assist in holding the diaper 10 to the legs of the individual to prevent leaking of fluids or other waste material from the diaper 10 by way of the leg openings 40 and 42.

Referring to FIGS. 1-5, in use, in one embodiment, the diaper 10 is positioned between the legs of an individual. The fasteners 50 are removably attached to adjust the rise of the diaper 10 to fit the diaper 10 to the appropriate size of the individual. The soaker 60 is positioned on the inner surface 18 of the diaper 10 such that the soaker 60 is between the inner surface 18 of the diaper 10 and the individual. The wings 24 and 28 are removably attached to one another and the wings 26 and 30 are removably attached to one another such that the diaper 10 is in the closed position and so that the diaper 10 is positioned on and removably attached to the individual.

One embodiment of the present invention is directed to a one-size-fits-all diaper, preferably, but not limited to, a reusable diaper that includes fasteners which adjust the size of the diaper to the individual. The diaper is reuseable over an extended period of time in a baby's/individual's growth. The diaper has fasteners used to adjust the rise/size of the diaper to the individual. As the individual grows, the fasteners are adjusted accordingly to the individual's size. One embodiment of the invention is directed to the adjustable snap-down feature of the diaper being positioned on the inner surface of the diaper. It should be understood that changes may be made in the operation and the setup of such embodiment.

From the above description, it is clear that the present invention is well-adapted to carry out the objects and to attain the advantages mentioned herein, as well as those inherent in the invention. While presently preferred embodiments of the invention have been described for purposes of this disclosure, it will be understood that numerous changes may be made which will readily suggest themselves to those skilled in the art and which are accomplished within the spirit of the invention disclosed.

What is claimed is:

1. A diaper, comprising:
a waist portion having a front portion and a back portion, the waist portion configured to be positioned about the waist of an individual; and
a crotch portion positioned between the front and back portions of the waist portion, the crotch portion configured to be positioned about the crotch of the individual, the crotch portion having an inner surface and an outer surface, the inner surface being provided with a plurality of fasteners so as to adjust a rise of the diaper to fit the individual.

2. The diaper of claim 1, wherein the front portion and the back portion of the waist portion are each provided with at least one fastener for removably attaching the front portion and the back portion of the waist portion so as to removably attach the waist portion to the individual.

3. The diaper of claim 1 wherein the front portion of the waist portion is provided with a pair of symmetrically disposed wings and the back portion of the waist portion is provided with a pair of symmetrically disposed wings.

4. The diaper of claim 3 wherein at least one fastener is provided to the pair of wings of the front portion of the waist portion and at least one fastener is provided to the pair of wings of the back portion of the waist portion so that the pair of wings of the front portion of the waist portion and the pair of wings of the back portion of the waist portion are removably attachable so as to removably attach the diaper to the individual.

5. The diaper of claim 4, wherein the at least one fastener is a snap.

6. The diaper of claim 1, wherein the plurality of fasteners are arranged in columns.

7. The diaper of claim 1, wherein at least two of the plurality of fasteners are color-coded to identify various range of sizes.

8. The diaper of claim 1, wherein the plurality of fasteners are snaps.

9. The diaper of claim 1, further comprising:
a soaker positioned along the inner surface of the diaper for absorbing fluid or waste material from the individual.

10. A method of adjusting a diaper, comprising the steps of:
providing the diaper having a waist portion having a front portion and a back portion, the waist portion configured to be positioned about the waist of an individual; and a crotch portion positioned between the front and back portions of the waist portion, the crotch portion configured to be positioned about the crotch of the individual, the crotch portion having an inner surface and an outer surface, the inner surface being provided with a plurality of fasteners; and
removably attaching at least two fasteners of the plurality of fasteners so as to adjust a rise of the diaper to fit the individual.

11. The method of claim 10, wherein the front portion and the back portion removably attaching the front portion and the back portion of the waist portion so as to removably attach the diaper to the individual.

12. The method of claim 10, further comprising:
providing the front portion of the waist portion with a pair of symmetrically disposed wings and the back portion of the waist portion with a pair of symmetrically disposed wings.

13. The method of claim 12 further comprising the step of:
removably attaching the pair of wings of the front portion of the waist portion and the pair of wings of the back portion of the waist portion so as to removably attach the waist portion to the individual.

14. The method of claim 10, further comprising the step of:
providing the plurality of fasteners arranged in columns.

15. The method of claim 10, further comprising the step of:
providing at least two of the plurality of fasteners as color-coded to identify various range of sizes.

16. The method of claim 10, further comprising:
positioning a soaker along the inner surface of the diaper for absorbing fluid or waste material from the individual.
